(19) United States
(12) Patent Application Publication
(10) Pub. No.: US 2012/0221047 A1
(43) Pub. Date: Aug. 30, 2012

(54) Chiropractic cervical traction-decompression device

(76) Inventors: Harvey J. Segur, Portland, OR (US); Sandra Kay Diehl, (US)

(21) Appl. No.: 12/932,363

(22) Filed: Feb. 24, 2011

(51) Int. Cl. A61F 5/00

(52) U.S. Cl. 606/241

(57) ABSTRACT

A portable device that provides a frame for cervical traction with an elevation clip holder that can produce gross or incremental adjustments along a vertical axis during traction supported by a stable rigid frame that can be attached to a manipulation or manipulative surgical table with support brackets, which is designed to be used in conjunction with a variety of cervical traction units and certain methods, techniques, and procedures to create decompression forces in the spine with the use of the hands of the doctor.

### CROSS-REFERENCE TO RELATED APPLICATIONS

Not Applicable

### FEDERALLY SPONSORED RESEARCH

Not Applicable

### SEQUENCE LISTING OF PROGRAM

Not Applicable

### BACKGROUND

1. Field

This application relates generally to improvements in cervical traction devices that are used in conjunction with the methods, techniques, and procedures of spinal manual manipulation and manipulative surgery by a doctor educated and trained in the professions of either chiropractic, osteopathy, naturopathy, or physical medicine for treatment of spinal and pelvic disorders, conditions, and syndromes.

2. Prior Art

Heretofore, there have been various devices that have been invented that claim to stretch, traction, decompress, mobilize, and reposition the articular elements of the cervical spine that have been designed for self-use, self treatment, or incorporated into a therapeutic table. The concept of the present invention is a portable device that provides cervical traction with an elevation clip holder that can produce gross or incremental adjustments during traction supported by a stable rigid frame that can be attached to a manipulation or manipulative surgical table with support brackets, which is designed to be used in conjunction with certain methods, techniques, and procedures to create decompression forces in the spine with the use of the hands of the doctor.

Prior art thought to be most closely associated with the present invention are the inventions disclosed in U.S. Pat. No. 1,374,115 to Roemer (1918), U.S. Pat. No. 2,821,978 to Lindstrom (1956), U.S. Pat. No. 3,033,198 to Jensen (1957), U.S. Pat. No. 3,118,442 to Montgomery (1964), U.S. Pat. No. 3,404,679 to Bevilacqua (1965), U.S. Pat. No. 3,420,229 to Miller (1966), U.S. Pat. No. 3,868,951 to Albrecht (1975), U.S. Pat. No. 4,257,410 to Flewelling (1981), U.S. Pat. No. 4,489,715 to Hall (1984), U.S. Pat. No. 4,508,109 (Re. 32,791) to Saunders (1988), and U.S. Pat. No. 4,890,605 to Rosendale (1990). The Roemer invention of 1918 is a therapeutic table designed to stretch and traction the spine under tension, but it does not have elevation adjustment capability nor is it portable. The Lindstrom invention of 1956 is a basic weighted prone traction device that has an adjustable external collar that must be fixed before traction, but is not capable of internal gross or incremental position adjustment during traction. The Jensen invention of 1957 is a motorized weighted prone traction device that has an adjustable external collar that must be fixed before traction, but is not capable of internal gross or incremental position adjustment during traction. The Montgomery invention of 1961 is an improvement of basic cervical or lumbar traction by providing a method to calculate therapeutic traction force and the amount of pounds pulled, but does not have the capacity of internal gross or incremental vertical position adjustment during traction. The Bevilacqua invention of 1965 is a medical therapy table capable of multi-positional cervical, lumbar and pelvic traction without requiring the patient to mount and dismount during treatment, but it does not have vertical cervical elevation adjustment capability nor is it portable. The Miller invention of 1966 is a motorized osteopathic therapy table designed to create rotatable synchronous movement of the spine in opposite direction with pin and hole prone cervical position traction capability, but is not capable of internal gross or incremental position adjustment during traction and is not portable. The Albrecht invention of 1975 is a portable motorized prone cervical traction device with calibrated side weights, however this invention is not capable of internal gross or incremental position adjustment during traction. The Flewelling invention of 1981 is an improvement of a portable cervical traction device designed to fit a variety of hospital beds with exterior elevation adjustment capability, but is not capable of internal gross or incremental position adjustment during traction. The Hall invention of 1984 is a portable cervical traction device designed to be used with the Well's tong attachment to stabilize a patient for transport and has external position adjustment, but is not designed to be used in conjunction with manual manipulation and does not have an internal gross or incremental position adjustment that can be used during treatment. The Saunders invention of 1988 is designed to apply tractile force to the occiput with two ‘V’ shaped arms that does have external position adjustment, but is not capable of internal gross or incremental position adjustment during traction and is not portable. The Rosendale invention of 1990 is a cervical traction device designed for transporting patients with spinal cord injury, but is not designed to be used in conjunction with manual manipulation and does not have an internal gross or incremental position adjustment that can be used during treatment.

None of the aforementioned inventions provide for a device that is designed to be used in conjunction with the methods, techniques, and procedures of spinal manual manipulation and manipulative surgery by a doctor educated and trained in the professions of either chiropractic, osteopathy, naturopathy, or physical medicine for the purpose of cervical traction-decompression.

### SUMMARY

The concept of the present invention is a portable device that provides a frame for cervical traction with an elevation clip holder that can produce gross or incremental adjustments along a vertical axis during traction supported by a stable rigid frame that can be attached to a manipulation or manipulative surgical table with support brackets, which is designed to be used in conjunction with a variety of cervical traction units and certain methods, techniques, and procedures to create decompression forces in the spine with the use of the hands of the doctor.

### DRAWINGS

The following is an itemized list of the figures referred to in this patent application.

FIG. 1 shows a perspective view of the cervical traction-decompression device.

### REFERENCE NUMBERS

1 Tube and channel.
2 Adjustment dial.
3 Base frame.
4 Attachment frame extension.
5 Knob handle.
6 Finger notch.
7 Clip holder.
8 Elevation adjustment rod.

### DETAILED DESCRIPTION OF PREFERRED EMBODIMENTS

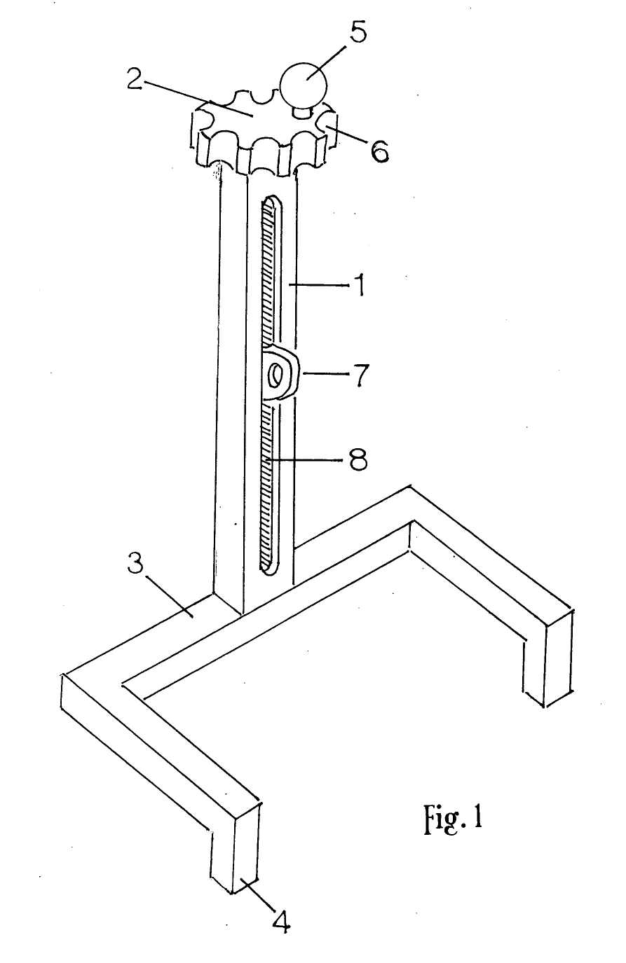

As shown in FIG. 1 a portable cervical traction device of the first embodiment consisting of a tube and channel 1 attached to a base frame 3 with attachment frame extension 4 that is used to secure the frame to a treatment table by brackets. The control of function of the cervical traction device consists of an adjustment dial 2 and knob handle 5 attached to an elevation adjustment rod 8 with a clip holder 7 that is capable of gross and incremental adjustments along a vertical axis up or down. The clip holder 7 is designed to allow for the attachment a variety of cervical traction units that are selected by the doctor. The cervical traction device frame and parts can be made of any rigid man-made material, plastic, or metal.

The scope of the invention should be determined by the appended claims and their legal equivalents and not by examples given.

1. A chiropractic cervical traction-decompression device consisting of
a. Tube and channel.
b. Adjustment dial.
c. Base frame.
d. Attachment frame extension.
e. Knob handle.
f. Finger notch.
g. Clip holder.
h. Elevation adjustment rod.
i. Portable

2. The concept of the first embodiment of claim 1 is a portable device that provides for a frame for cervical traction with an elevation clip holder that can produce gross or incremental adjustments during traction along a vertical axis supported by a stable rigid frame that can be attached to a manipulation or manipulative surgical table with support brackets.

3. A device of claim 1 which is designed to be used in conjunction with certain methods, techniques, and procedures to create decompression forces in the spine with the use of the hands of the doctor.

4. A device of claim 1 that can produce traction in 360 degrees when combined with the patient position of prone, supine, side recumbent, or a variation of such positions.

5. A function of the device of claim 1 using clip holder 7 is designed to allow for the attachment a variety of cervical traction units that are selected and used by the doctor.
